(19) United States
(12) Patent Application Publication
(10) Pub. No.: US 2003/0225446 A1
(43) Pub. Date: Dec. 4, 2003

(54) Multi-piece prosthesis deployment apparatus

(75) Inventor: David Ernest Hartley, Subiaco (AU)

Correspondence Address: COOK GROUP PATENT OFFICE, P.O. BOX 2269, BLOOMINGTON, IN, 47402

(73) Assignee: William A. Cook Australia Pty Ltd., Brisbane (AU)

(21) Appl. No.: 10/447,380

(22) Filed: May 29, 2003

Related U.S. Application Data
(60) Provisional application No. 60/384,056, filed on May 29, 2002.

(51) Int. Cl. A61F 2/06

(52) U.S. Cl. 623/1.11; 606/108

(57) ABSTRACT

A multi-piece prosthesis deployment apparatus comprising a longitudinally extending inner body arrangement, an outer deployment sheath arrangement, and axially spaced regions extending between the body arrangement and the sheath, each region serving to contain a respective prosthesis for sequential deployment within a lumen of a patient.

### CROSS-REFERENCE TO RELATED APPLICATIONS

This application claims priority to U.S. provisional application Serial No. 60/384,056, filed May 29, 2002, of the same title.

### TECHNICAL FIELD

This invention relates to devices and apparatus for the deployment of a multi-piece prosthesis such as an endoluminal stent graft and will be particularly discussed in relation to its application for deployment of such a stent graft into the aorta of a patient for treatment of abdominal aortic aneurysm, although the invention is not limited to this application.

### BACKGROUND OF THE INVENTION

Throughout this specification the term “distal” with respect to a prosthesis or a prosthesis deployment device is the end of the prosthesis which is furthest away in the direction of blood flow from the heart, and the term “proximal” means the end of the prosthesis or prosthesis deployment device which is nearest to the heart. The same terminology will be used in relation to the aorta or other lumen. It will be realized that for other body lumens then corresponding terminology such as “cranial” and “caudal” should be understood.

In some circumstances, it is desirable to introduce or deploy a two or more piece prosthesis into a patient's lumen and this can be done in two separate steps using two deployment devices, each with a prosthesis carried on the deployment device. It may be less traumatic to a patient, however, if deployment can be achieved using a single deployment device.

It is the object of this invention to provide such a device or at least to provide a useful alternative.

### SUMMARY OF THE INVENTION

In one form therefore, the invention is said to reside in a prosthesis deployment apparatus comprising a longitudinally extending inner body arrangement, an outer deployment sheath arrangement, and axially spaced regions extending between the body arrangement and the sheath, each region serving to contain a respective prosthesis for sequential deployment within a lumen of a patient.

In a preferred embodiment, the inner body arrangement comprises a catheter arrangement extending along the apparatus, and wherein each said region extends between either the outer surface of the catheter and the sheath, or between a catheter body portion and the sheath.

Preferably the regions are axially separated by a catheter body part or portion.

In a further form, the invention is said to reside in a multi-piece prosthesis deployment apparatus comprising:

a deployment catheter having a proximal end adapted to be introduced into a patient and a distal end adapted to remain outside a patient, the catheter having at least a first proximal annular region adapted in use to contain a first prosthesis and a second annular region distal of the first annular region, the second annular region adapted in use to contain a second prosthesis, and a sheath arrangement adapted in use to extend over and cover the first region and the second region and adapted to be moved with respect to the catheter to sequentially expose the first region and the second region to thereby enable deployment of the first prosthesis and then the second prosthesis.

In a further form, the invention is said to reside in a multi-piece prosthesis deployment apparatus including a catheter having a proximal end adapted to be introduced into a patient and a distal end adapted to remain outside a patient, the catheter having at least a first proximal annular region for containing a first prosthesis and a second annular region distal of the first annular region, the second annular region for containing a second prosthesis and a sheath arrangement extending over and covering the at least first region and the second region, the sheath arrangement adapted to be moved with respect to the catheter to sequentially expose the first region and to allow deployment of the first prosthesis and then the second region to allow deployment of the second prosthesis.

It will be seen that by the various forms of the invention there is provided an arrangement in which at least two prostheses can be carried into the body of a patient to a lumen requiring grafting and by sequential deployment the first prosthesis and then the second prosthesis can be deployed.

Preferably the proximal end of the catheter has a long flexible tapered nose cone to assist with insertion of the catheter through arteries to the site of deployment.

The regions can be part of one continuous region and the two prostheses can be next to one another or they could be spaced apart. The inner body arrangement can include a catheter body having annular recesses as the respective regions.

There may be provided a central guide wire lumen along the length of the catheter so that a guide wire may be first inserted into a patient and the deployment device deployed along the guide wire with the guide wire passing through the central guide wire lumen to assist with placement in the correct location.

The central or guide wire lumen may include a guide wire catheter with the guide wire catheter coaxial with the guide wire lumen and able to move longitudinally therealong.

The guide wire catheter may extend through a lumen in the catheter so that the guide wire catheter can move longitudinally and rotationally with respect to the catheter.

The deployment apparatus according to this invention may be adapted for the supply of contrast fluids through the guide wire lumen or catheter. For this purpose there may be provided contrast fluid ports in the flexible nose cone and the lumen or catheter extending to the flexible nose cone and a syringe socket on the central lumen or catheter at the distal end of the deployment catheter to enable the contrast fluid to be added.

Preferably the sheath terminates distally in a sealing assembly to seal against the catheter at a position outside the patient and the sheath termination may include a contrast fluid supply point to enable an injection of contrast fluid between the sheath and catheter. This will enable visualization of the point of deployment of the second prosthesis as will be discussed with respect to the preferred embodiment.

In a preferred form of the invention the first or proximal region may include a retention arrangement for retaining the proximal end of the first prosthesis. The retention arrangement may include a trigger wire to release the prosthesis when the prosthesis has been positioned in the correct place. The retention arrangement may be positioned just distal of the nose cone and move with the nose cone.

There may be further provided a retention arrangement for the distal end of the first prosthesis so that after the proximal end of the prosthesis has been deployed, the distal end of the prosthesis is retained with respect to the catheter and hence may be manipulated by manipulation of the catheter. Such manipulation may be used to shorten, lengthen or twist the prosthesis. The retention arrangement for the distal end of the first prosthesis may use the same or a separate trigger wire to release the distal end of the prosthesis when the prosthesis is correctly deployed.

Preferably the trigger wire extends from the outside of the patient where it is retained by a trigger wire release mechanism on a handle at the distal end of the catheter. The trigger wire extends in the annular lumen between the catheter and central guide wire catheter and in the first region may extend through a lumen formed on the outside of the central guide wire catheter. It will be realized therefore, that by this arrangement during deployment of the first or more proximal prosthesis, the proximal end of the prosthesis can be correctly positioned and deployed and then sequentially the distal end of the first prosthesis may be correctly positioned and deployed. For this purpose the sheath arrangement is preferably adapted for partial retraction and guide markings may be provided on the catheter outside the patient to enable a surgeon to correctly deploy the sheath by the right amount.

To assist deployment of the proximal prosthesis, the nose cone portion of the deployment catheter may be adapted to be moved longitudinally and rotated with respect to the catheter body to enable accurate placement of the proximal prosthesis. To enable this action, the nose cone portion may be mounted on the central guide wire catheter to be rotated or moved longitudinally by movement of the central guide wire catheter with respect to the deployment catheter. A locking or clamping arrangement may be provided to fix the position of the central guide wire catheter with respect to the deployment catheter. This may be in the form of a pin vice.

In one form of the invention, the distal or second prosthesis retained in the second region does not have any form of retention other than by being compressed by the sheath arrangement until the sheath arrangement is withdrawn to deploy the second prosthesis.

Once again, the deployment of the distal or second prosthesis may be a sequential process where the sheath arrangement is partially withdrawn to expose the proximal end of the distal prosthesis and when this has been correctly positioned, the deployment catheter can be manipulated with the distal end of the second prosthesis still retained within the sheath arrangement until its correct position is obtained.

In an alternative embodiment, the second prosthesis may include proximal and/or distal retention arrangements with respective trigger wires as explained with respect to the first prosthesis. This trigger wire may use the same trigger wire release mechanism or there may be a further trigger wire release mechanism.

Preferably each prosthesis is of a self-expanding type using zigzag Z stents or other self-expanding stents mounted onto or into a tube of biocompatible graft material to enable it to bear against the walls of the lumen into which the prosthesis is deployed to provide a good seal. In a preferred form of the invention, at least the proximal prosthesis may have zigzag Z stents extending from the proximal end adapted to engage against the walls of the lumen. These proximally extending zigzag Z tents may include barbs to engage into the wall of the lumen.

The tube of bio-compatible graft material can include polytetrafluoroethylene, Dacron, polyamide or any other suitable biocompatible graft material.

While DACRON, expanded polytetrafluoroethylene (ePTFE), or other synthetic biocompatible materials can be used for the tubular graft material for the stent graft, a naturally occurring biomaterial such as collagen, is highly desirable, particularly a specially derived collagen material known as an extracellular matrix (ECM), such as small intestinal submucosa (SIS). Besides SIS, examples of ECM's include pericardium, stomach submucosa, liver basement membrane, urinary bladder submucosa, tissue mucosa, and dura mater.

SIS is particularly useful, and can be made in the fashion described in Badylak et al., U.S. Pat. No. 4,902,508; Intestinal Collagen Layer described in U.S. Pat. No. 5,733,337 to Carr and in 17 Nature Biotechnology 1083 (November 1999); Cook et al., WIPO Publication WO 98/22158, dated May 28, 1998, which is the published application of PCT/US97/14855, the teachings of which are incorporated herein by reference. Irrespective of the origin of the material (synthetic versus naturally occurring), the material can be made thicker by making multilaminate constructs, for example SIS constructs as described in U.S. Pat. Nos. 5,968,096; 5,955,110; 5,885,619; and 5,711,969. In addition to xenogenic biomaterials such as SIS, autologous tissue can be harvested as well, for use in forming the tubular graft material. Additionally Elastin or Elastin-Like Polypeptides (ELPs) and the like, offer potential as a material to fabricate the tubular graft material to form a device with exceptional biocompatibility.

SIS is available from COOK Biotech, West Lafayette, Ind., USA.

PCT Patent Publication No. WO 98/53761 entitled “A Prosthesis and a Method of Deploying a Prosthesis” discloses an introducer for a prosthesis which retains the prosthesis so that each end can be moved independently. These features and other features disclosed in PCT Patent Publication No. WO 98/53761 could be used with the present invention and the disclosure of PCT Patent Publication No. WO 98/53761 is herewith incorporated in its entirety into this specification.

PCT Patent Application No. PCT/US02/34348 entitled “Prostheses for Curved Lumens” discloses prostheses with arrangements for bending the prosthesis for placement into curved lumens. This feature and other features disclosed in PCT Patent Application No. PCT/US02/34348 could be used with the present invention, and the disclosure of PCT Patent Application No. PCT/US02/34348 is herewith incorporated in its entirety into this specification.

U.S. Pat. No. 6,206,931 entitled “Graft Prosthesis Materials” discloses graft prosthesis materials and a method for implanting, transplanting, replacing and repairing a part of a patient and particularly the manufacture and use of a purified, collagen based matrix structure removed from a submucosa tissue source. These features and other features disclosed in U.S. Pat. No. 6,206,931 could be used with the present invention, and the disclosure of U.S. Pat. No. 6,206,931 is herewith incorporated in its entirety into this specification.

Australian Provisional Patent Application No. PS3215 entitled “A Stent Graft Fastening Arrangement” discloses arrangements for fastening stents onto grafts particularly for exposed stents. This feature and other features disclosed in Australian Provisional Patent Application No. PS3215, could be used with the present invention, and the disclosure of Australian Provisional Patent Application No. PS3215 is herewith incorporated in its entirety into this specification.

Australian Provisional Patent Application No. PR9617 entitled “Improving Graft Adhesion” discloses arrangements on stent grafts for enhancing the adhesion of such stent grafts into walls of vessels in which they are deployed. This feature and other features disclosed in Australian Provisional Patent Application No. PR9617, could be used with the present invention and the disclosure of Australian Provisional Patent Application No.

PR9617 is herewith incorporated in its entirety into this specification.

### BRIEF DESCRIPTION OF THE DRAWING

This then, generally describes the invention, but to assist with understanding, reference will now be made to the accompanying drawings which show preferred embodiments of the invention and the method of using the device of the present invention.

In the drawings:

FIG. 1 shows a general view of an embodiment of a two piece graft deployment apparatus according to the invention;

FIG. 2 shows detail of the recess region of the deployment device shown in FIG. 1;

FIGS. 3A, 3B and 3C show more detail of the deployment device;

FIGS. 4A to 4G show the various stages of the deployment of a graft using the device of the present invention;

FIG. 5 shows detail of the recess region of an alternative embodiment of a deployment device according to the invention; and

FIGS. 6A, 6B and 6C show more detail of the embodiment shown in FIG. 5.

### DETAILED DESCRIPTION

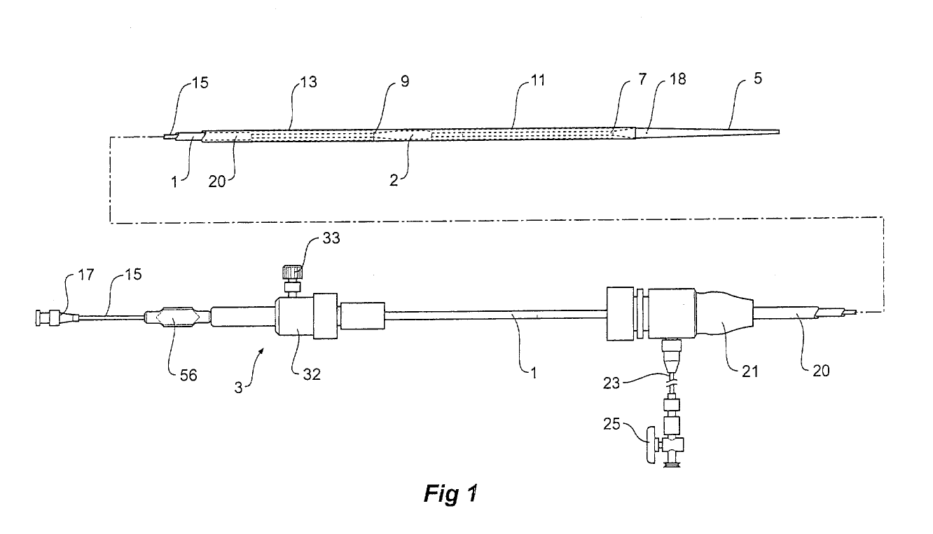

Now looking more closely at the drawings, and in particular, a first embodiment shown in FIGS. 1 to 3C, it will be seen that the prosthesis deployment apparatus or device of this invention comprises a catheter 1 and catheter extension 2 extending between a distal end 3 including a handle portion 4 adapted in use to remain outside the body of a patient and a nose cone 5 adapted in use to be inserted through the arteries of a patient. For treatment of an aortic aneurism, for instance, the deployment apparatus or device is inserted through a femoral artery and into the iliac arteries and then into the aorta. There are two regions in the catheter body being a proximal region 7 and a distal region 9. The proximal region is adapted to retain a proximal prosthesis 11 and the distal region is adapted to retain a distal prosthesis 13.

The hollow central guide wire catheter 15 extends from a distal syringe attachment point 17 through a lumen in the catheter 1 and catheter extension 2 to the nose cone 5 so that manipulation of the central catheter 15 with respect to the catheter body 1 will move the nose cone 5 with respect to the catheter body 1. Manipulation can be either rotation or longitudinal movement.

A pin vice 56 at the distal end of the deployment catheter 1 locks the position of the central catheter 15 with respect to the deployment catheter 1.

The catheter extension 2 is mounted to the catheter 1 by an extension 16 and moves with it, and the guide wire catheter 15 is coaxial within the extension 16 and can move with respect to it.

The catheter body is surrounded by a sheath 20 extending from a sheath termination hub or point 21 forward to the nose cone 5. The sheath termination 21 includes a side tube 23 with a hypodermic syringe attachment point 25 so that contrast fluid can be supplied into the sheath termination hub to travel between the sheath 20 and the catheter body 1 as will be discussed later.

The hypodermic syringe connection point 17 is adapted for the supply of contrast fluid through the central guide wire catheter 15 to the nose cone 5 where it is adapted to be ejected through ports 18 in the sides of the nose cone.

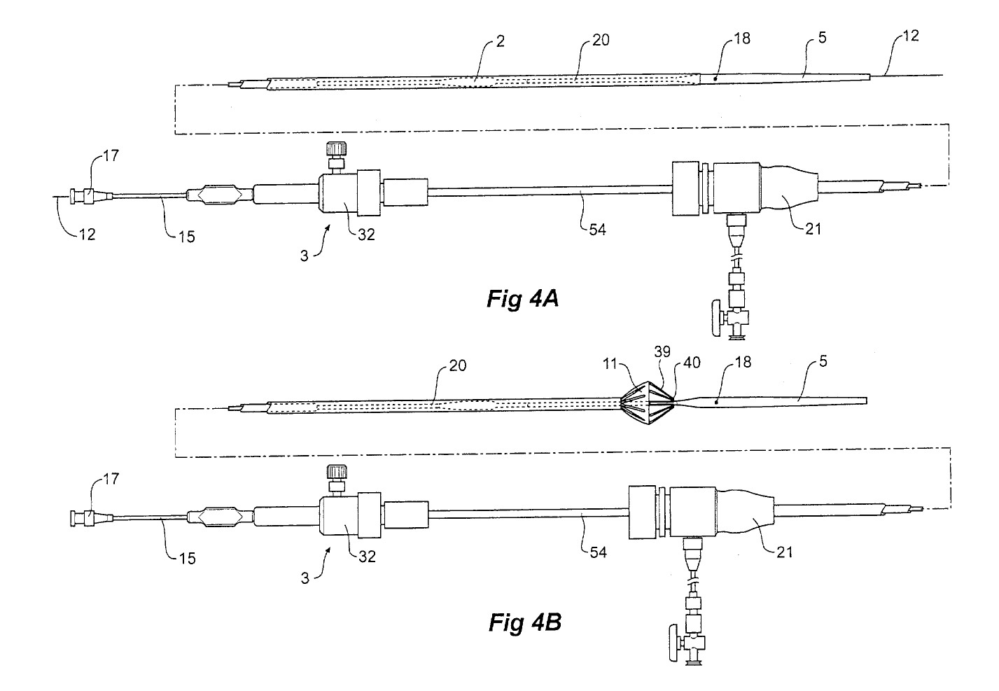

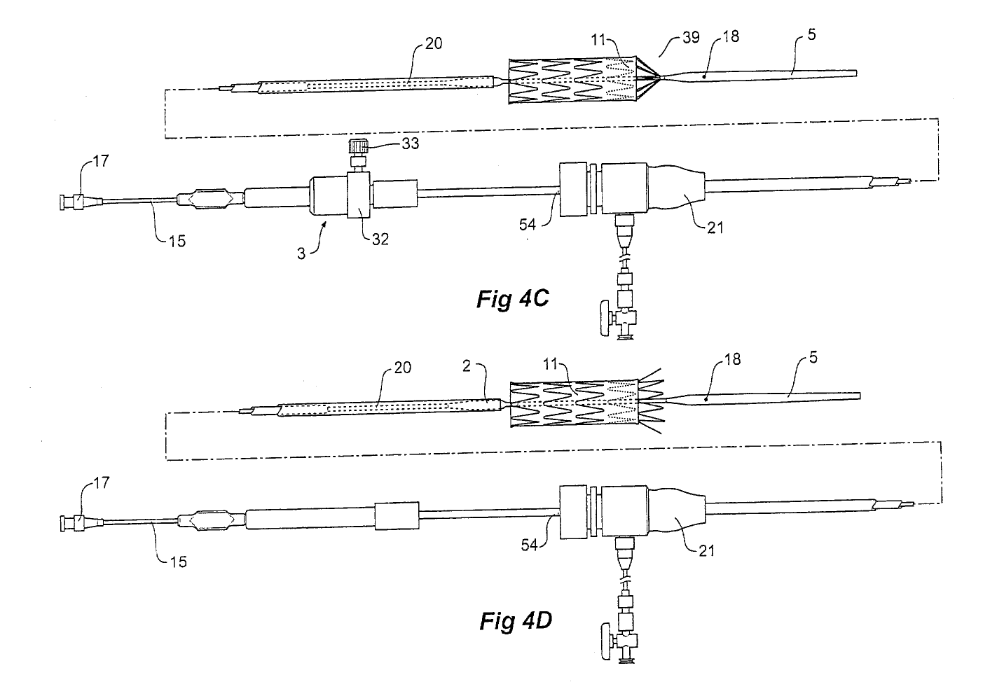

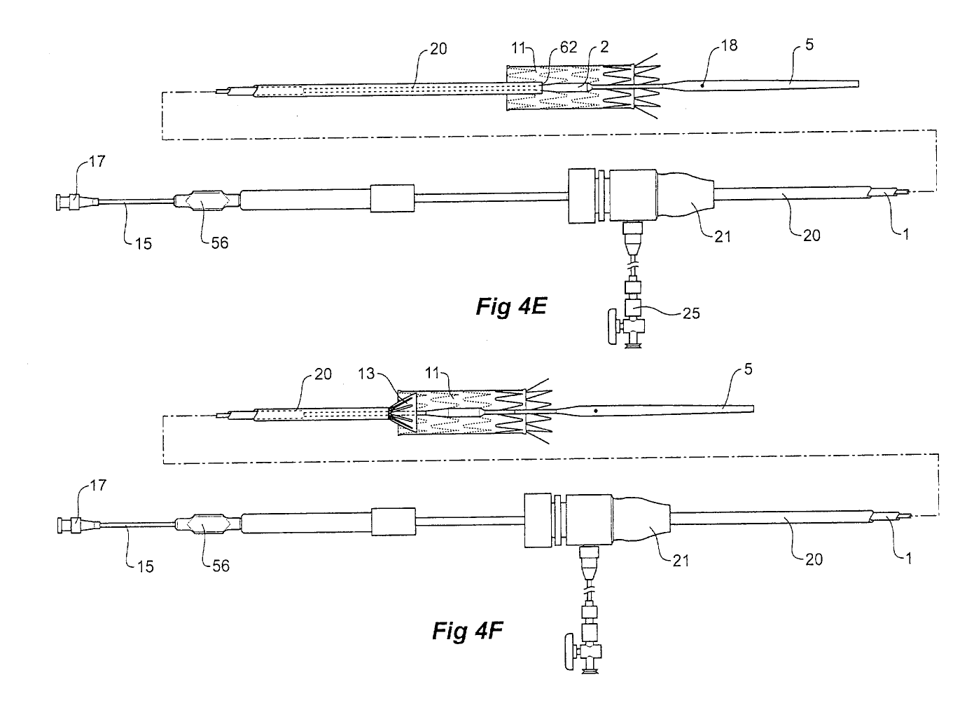

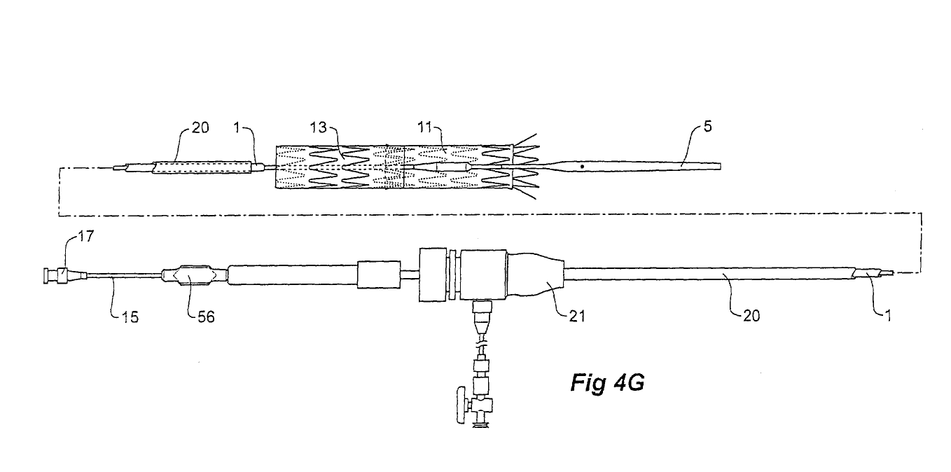

A trigger wire 30 is deployed in the lumen between the central guide wire 15 catheter and the catheter body 1 and extends from a trigger wire release mechanism 32 on the handle 4 at the distal end of the catheter 1 to the proximal end of the proximal prosthesis 11. The trigger wire release mechanism 32 has a thumb screw 33 which can be rotated to release the trigger wire retention mechanism 32 which can then be removed as will be discussed with respect to the various stage shown in FIG. 4.

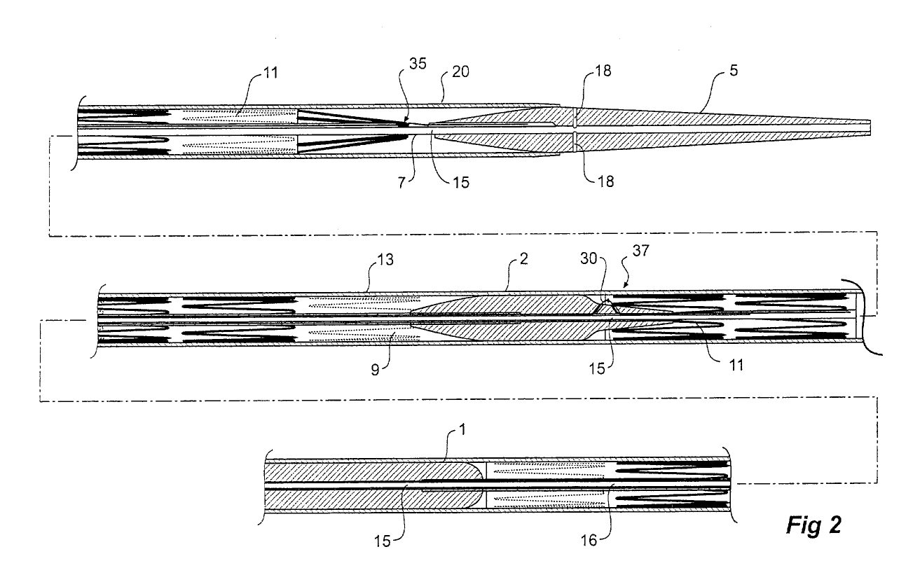

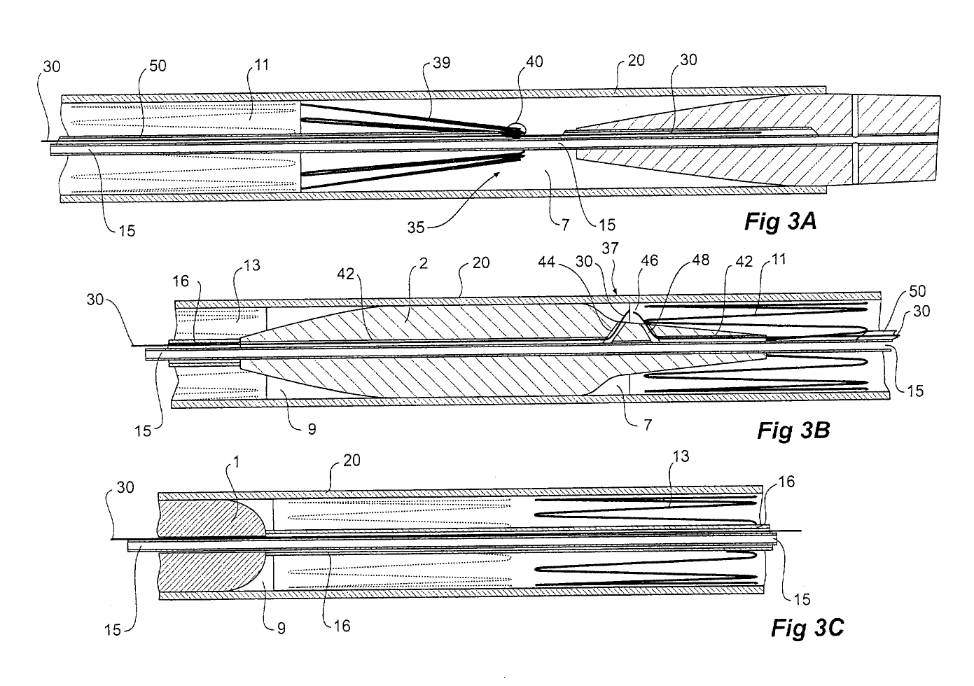

Now looking more closely at the detailed drawing FIG. 2 and FIGS. 3A, 3B and 3C, it will be seen that the distal or second prosthesis 13 is retained in the second region 9 by being compressed by the sheath 20 but that no other retention arrangement is provided.

The proximal or first prosthesis 11 is retained in the first region 7 both by the sheath 20 and by a proximal retention arrangement generally shown as 35 and a distal release arrangement generally shown as 37. The first prosthesis 11 has zigzag stents 39 extending from its proximal end and these are pulled together by a mooring loop 40 which is retained by the trigger wire 30. Upon withdrawal of the trigger wire 30, the mooring loop releases the ends of the zigzag stent 39 as will be discussed with respect to FIG. 4C. At this stage the mooring loop can be adapted to be retained on the central catheter 15 to be withdrawn with the deployment arrangement after deployment of the prosthesis or it can remain with the zigzag stents. The distal retention arrangement 37 for the first prosthesis includes the same trigger wire 30 which exits the lumen 42 between the guide wire catheter 15 and the catheter extension 2 through aperture 44 and then passes through the distal end of the graft at 46 before passing through another aperture 48 back into the lumen 42.

Proximally of the catheter extension 2, a trigger wire lumen 50 is formed on the outside of the central guide wire catheter 15 and this trigger wire lumen 50 extends to the proximal end of the recess 7. At this point the trigger wire engages the mooring loop 40 as discussed earlier.

The sequence of deployment of a two-piece prosthesis using the deployment device of the present invention will be discussed with respect to FIGS. 4A to 4G.

FIG. 4A shows the device in its loaded ready to deploy configuration. The deployment apparatus or device according to this invention is inserted over a guide wire 12, which is inserted through the central catheter 15 until the nose cone 5 is substantially in the region where the prostheses are to be deployed. The guide wire can then be removed. A hypodermic syringe is then connected to the syringe connection point 17 and contrast media ejected out through the ports 18 on the nose cone and observed by x-ray fluoroscopy or angiography. By this arrangement, the deployment apparatus or device can be positioned so that the proximal prosthesis 11 is in the required place.

The sheath 20 is then withdrawn by grasping and pulling on the sheath termination 21 until the proximal or first prosthesis is exposed as shown in FIG. 4B, but the proximally extending zigzag stents 39 are still retained by the mooring loop 40 engaged with the trigger wire 30. The position of the zigzag stents and the proximal end of the proximal graft 11 can then be visualized by x-ray fluoroscopy to ensure that it is in the correct position.

The sheath 20 is then withdrawn to the marking 54 on the catheter body 1 by pulling on the sheath termination hub 21. At this stage, the sheath still covers the catheter extension 2, but the proximal prosthesis 11 is deployed but retained at the proximal end by the proximally extending zigzag stent 39, which is still retained by the mooring loop 40 engaged with the trigger wire 30 at the distal retention point 37 by the trigger wire. This position is shown in FIG. 4C.

When any final correction of the position is achieved, the trigger wire release thumb screw 33 is released and the trigger release 32 is moved back enough to release to the proximal end of the graft. At this stage the proximally extending zigzag stent 39 fans out to engage the walls of the graft as shown in FIG. 4D, and if present, the barbs thereon engage into the wall to fix that end of the prosthesis. Next, the trigger wire release mechanism 32 is completely removed so that the proximal prosthesis is fully deployed.

At this stage, the entire deployment apparatus is advanced further into the artery so that the proximal end of the second prosthesis is within the distal end of the first prosthesis. This position is shown in FIG. 4E. If desired, the nose cone 5 can be retracted by releasing the pin-vice 56 and withdrawing the central catheter 15.

At this stage, a contrast medium may be injected through the port 25 into the sheath termination 21 so that contrast fluid travels up between the sheath 20 and the catheter body 1 to exit in the region shown as 62 in FIG. 4E. This enables the position of the second prosthesis to be determined with respect to the first prosthesis before withdrawal of the sheath as shown in FIG. 4F.

The sheath 20 is then further withdrawn up until the proximal end of the second graft 13 is released and this expands so that it engages on the inner surface of the first graft 11. This is shown in FIG. 4F.

The sheath 20 is then further withdrawn so that the second prosthesis is completely deployed and the sheath 20 is withdrawn over the catheter body 1. This is shown in FIG. 4G.

The sheath arrangement or assembly can be then advanced so that it extends to the nose portion 5, the position it is shown in FIG. 4A, and then the entire arrangement or assembly can be withdrawn.

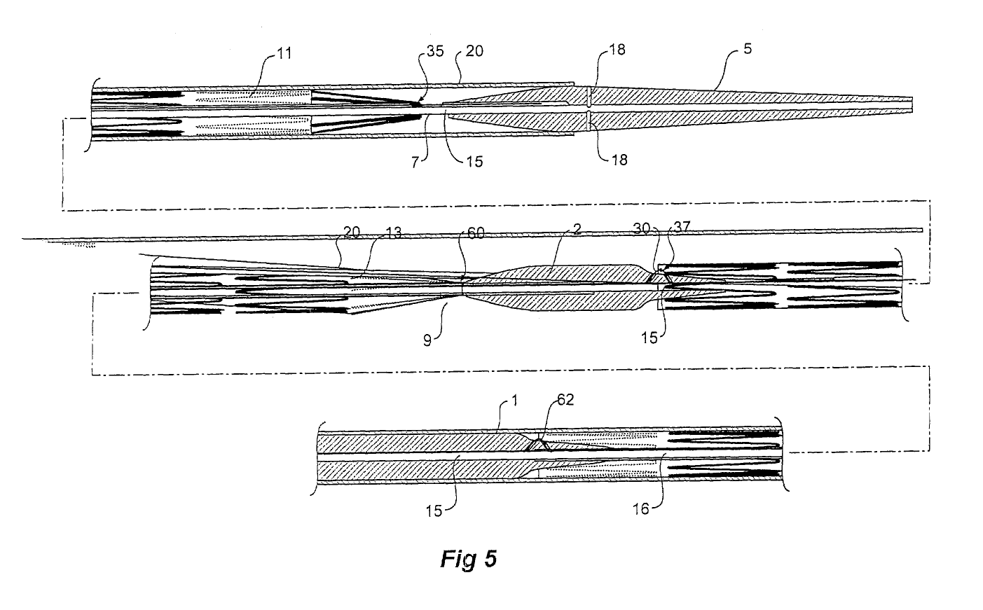

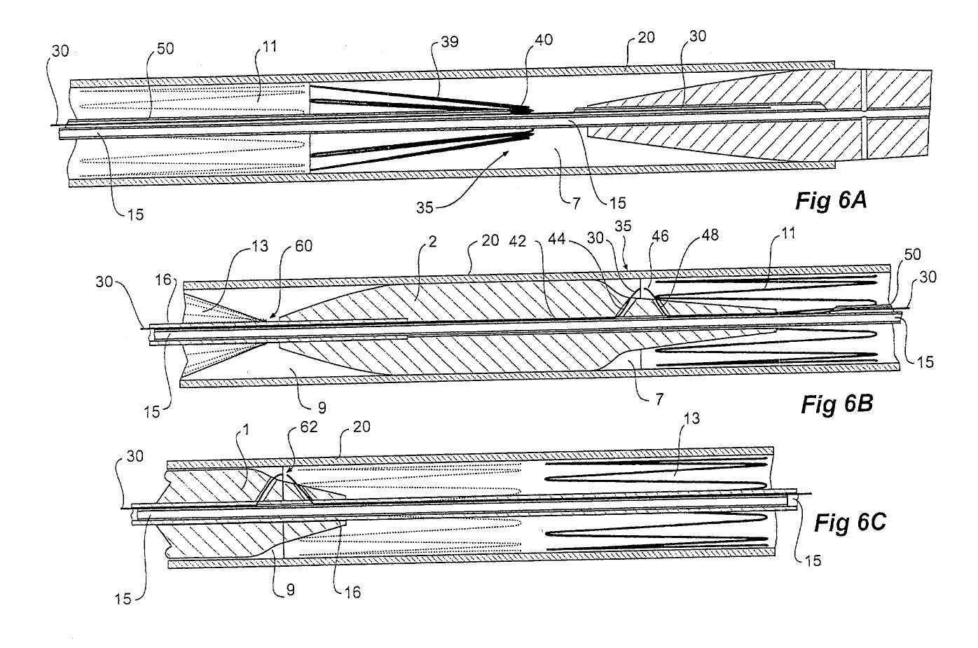

FIGS. 5 and 6A to 6C shows detail of the prosthesis retention regions of an alternative embodiment of a deployment apparatus or device according to the present invention. Those components with the same function as in the first embodiment have been given the same reference numerals.

In this embodiment it will be seen that the proximal or first prosthesis 11 is retained in the first region 7 both by the sheath 20 and by a proximal retention arrangement generally shown as 35 and a distal release arrangement generally shown as 37 as in the earlier embodiment.

In this embodiment, in a similar manner to the proximal prosthesis, the distal prosthesis is retained not only by the sheath 20 but also by a proximal retention arrangement generally shown as 60 and a distal release arrangement generally shown as 62. These enable the sheath 20 to be withdrawn independently of the full deployment of the distal prosthesis which may be of advantage in some situations. The trigger wire or wires for the distal prosthesis may be the same as or in addition to the trigger wires for the proximal prosthesis.

Throughout this specification various indications have been given as to the scope of the invention but the invention is not limited to any one of these but may reside in two or more of these combined together. The examples are given for illustration only and not for limitation.

What is claimed is:

1. A multi-piece prosthesis deployment apparatus comprising a longitudinally extending inner body arrangement, an outer deployment sheath arrangement, and axially spaced regions extending between the body arrangement and the sheath arrangement, each region serving to contain a respective prosthesis for sequential deployment within a lumen of a patient.

2. A multi-piece prosthesis deployment apparatus as in claim 1 wherein the inner body arrangement comprises a catheter arrangement extending along the apparatus, and wherein each said region extends between either the outer surface of the catheter and the sheath, or between a catheter body portion and the sheath.

3. A multi-piece prosthesis deployment apparatus as in claim 1 wherein the regions are axially separated by a catheter body part or portion.

4. A multi-piece prosthesis deployment apparatus comprising:
a deployment catheter having a proximal end adapted to be inserted into a patient and a distal end adapted to remain outside a patient, the catheter having a first proximal annular region adapted in use to contain a first prosthesis and a second annular region distal of the first annular recess, the second annular region adapted in use to contain a second prosthesis, and a sheath arrangement adapted in use to extend over and cover the first region and the second region and adapted to be moved with respect to the catheter to sequentially expose the first region and the second region to thereby enable deployment of the first prosthesis and then the second prosthesis.

5. A multi-piece prosthesis deployment apparatus comprising:
a catheter having a proximal end adapted to be inserted into a patient and a distal end adapted to remain outside a patient, the catheter having at least a first proximal annular region for containing a first prosthesis and a second annular region distal of the first annular region, the second annular region for containing a second prosthesis; and
a sheath arrangement extending over and covering the at least first region and the second region, the sheath arrangement adapted to be moved with respect to the catheter to sequentially expose the at least first region and to allow deployment of the first prosthesis and then to expose the second region to allow deployment of the second prosthesis.

6. A multi-piece prosthesis deployment apparatus as in claim 5 further including a central lumen along the length of the catheter so that a guide wire may be first inserted into a patient and the deployment apparatus deployed along the guide wire with the guide wire passing through the central lumen to assist with placement in the correct location.

7. A multi-piece prosthesis deployment apparatus as in claim 6 wherein the central lumen includes a guide wire catheter.

8. A multi-piece prosthesis deployment apparatus as in claim 7 wherein the proximal end of the catheter has a long flexible tapered nose to assist with insertion of the catheter through arteries to the site of deployment.

9. A multi-piece prosthesis deployment apparatus as in claim 8 wherein the guide wire catheter has contrast fluid ports in the flexible nose and a syringe socket on the guide wire catheter at the distal end adapted for the supply of contrast fluids through the guide wire catheter.

10. A multi-piece prosthesis deployment apparatus as in claim 5 wherein the sheath terminates distally in a sealing assembly to seal against the catheter at a position outside the patient and the sheath termination includes a contrast fluid supply point to enable an injection of contrast fluid between the sheath and catheter whereby to enable visualization of the point of deployment of the second prosthesis.

11. A multi-piece prosthesis deployment apparatus as in claim 5 wherein the first or proximal region includes a retention arrangement for retaining the proximal end of the first prosthesis.

12. A multi-piece prosthesis deployment apparatus as in claim 11 wherein the retention arrangement includes a trigger wire to release the first prosthesis when the first prosthesis has been positioned in the correct place.

13. A multi-piece prosthesis deployment apparatus as in claim 12 further including a retention arrangement for the distal end of the first prosthesis whereby after the proximal end of the first prosthesis has been deployed, the distal end of the first prosthesis may be manipulated by manipulation of the catheter.

14. A multi-piece prosthesis deployment apparatus as in claim 13 wherein the retention arrangement for the distal end of the prosthesis uses the trigger wire or a further trigger wire to release the distal end of the prosthesis when the prosthesis is correctly deployed.

15. A multi-piece prosthesis deployment apparatus as in claim 13 wherein the trigger wire extends from the outside of the patient at a trigger wire release mechanism on a handle at the distal end of the catheter.

16. A multi-piece prosthesis deployment apparatus as in claim 13 wherein in the first region the trigger wire runs in a lumen of a catheter outside of a central guide wire catheter.

17. A multi-piece prosthesis deployment apparatus as in claim 5 wherein the sheath arrangement is adapted for partial retraction and guide markings are provided on the catheter outside the patient to enable a surgeon to correctly deploy the sheath by the right amount.

18. A multi-piece prosthesis deployment apparatus as in claim 5 wherein a nose portion of the catheter is adapted to be moved longitudinally and rotated with respect to the catheter body to enable accurate placement of the proximal prosthesis.

19. A multi-piece prosthesis deployment apparatus as in claim 18 wherein the nose portion is mounted on a central guide wire catheter to be rotated or moved longitudinally by movement of the guide wire catheter with respect to the catheter and a locking or clamping arrangement is provided to fix the position of the guide wire catheter with respect to the catheter.

20. A multi-piece prosthesis deployment apparatus as in claim 19 wherein the locking or clamping arrangement is a pin vice.

21. A multi-piece prosthesis deployment apparatus as in claim 5 wherein the distal or second prosthesis retained in the second region does not have any form of retention other than by being compressed by the sheath arrangement until the sheath arrangement is withdrawn to deploy the second prosthesis.

22. A multi-piece prosthesis deployment apparatus as in claim 5 wherein the distal or second prosthesis includes proximal and/or distal retention arrangements with respective trigger wires.

23. A multi-piece prosthesis deployment apparatus as in claim 5 wherein each prosthesis is of a self-expanding type using zigzag Z stents or other self-expanding stents to enable to bear against the walls of the lumen into which the prosthesis is deployed to provide a good seal.

24. A multi-piece prosthesis deployment apparatus as in claim 5 wherein the second prosthesis has zigzag Z stents extending from the proximal end adapted to engage against the walls of the lumen.

25. A multi-piece prosthesis deployment apparatus as in claim 24 wherein the proximally extending zigzag Z stents include barbs to engage into the wall of the lumen.
